(19) United States
(12) Patent Application Publication
(10) Pub. No.: US 2023/0018374 A1
(43) Pub. Date: Jan. 19, 2023

(54) LIGHTWEIGHT, MOBILE 3D FACE IMAGING SYSTEM FOR CLINICAL ENVIRONMENTS

(71) Applicant: UNIVERSITY OF SOUTHERN CALIFORNIA, LOS ANGELES, CA (US)

(72) Inventors: Wael ABD-ALMAGEED, Woodstock, MD (US); Leonidas SPINOULAS, Los Angeles, CA (US)

(73) Assignee: UNIVERSITY OF SOUTHERN CALIFORNIA, LOS ANGELES, CA (US)

(21) Appl. No.: 17/865,290

(22) Filed: Jul. 14, 2022

Related U.S. Application Data
(60) Provisional application No. 63/221,639, filed on Jul. 14, 2021.

(51) Int. Cl. G06T 7/00; H04N 5/225; H04N 5/232

(52) U.S. Cl. CPC G06T 7/0012; H04N 5/2256; H04N 5/232933; G06T 2207/20081; G06T 2207/20084; G06T 2207/30004

(57) ABSTRACT

A camera system for collecting image data in a clinical environment includes a camera configured to acquire color (RGB), near-infrared, and depth image data and a plurality of LED modules held proximate to the camera such that the LED modules are configured to illuminate a subject while image data is collected by the camera. The camera system also includes a microcontroller in electrical communication with the camera and the plurality of LED modules. The microcontroller is configured to actuate the plurality of LED modules and the camera such that the camera acquires the image data. A computing device is in electrical communication with the camera and the microcontroller.

### CROSS-REFERENCE TO RELATED APPLICATIONS

This application claims the benefit of U.S. provisional application Ser. No. 63/221,639 filed Jul. 14, 2021, the disclosure of which is hereby incorporated in its entirety by reference herein.

### TECHNICAL FIELD

In at least one aspect, a camera system that can be used system to collect image data in a clinical environment is provided.

### BACKGROUND

Classical congenital adrenal hyperplasia (CAH) is the most common cause of primary adrenal insufficiency in children, affecting one in 15,000 individuals. CAH is identified through universal newborn screening in the U.S. CAH is also a disorder of androgen excess secondary to disrupted steroid biosynthesis, with androgen overproduction from the adrenal glands beginning in week seven of fetal life. This prenatal androgen exposure and cortisol deficiency likely represent a significant change to the intrauterine environment during early human development, that could adversely impact fetal programming and thereby change the risk for later-life disease. Females with CAH are especially affected by prenatal androgen exposure, exhibiting masculinized external genitalia at birth and male-typical behaviors in childhood. Brain structural abnormalities have also been identified in youth and adults with CAH (smaller whole brain, prefrontal cortex, and medial temporal lobe volumes), along with concerning adverse cardiometabolic and neuropsychological outcomes over the lifespan of CAH individuals including obesity, hypertension, and a heightened potential for psychiatric disorders, substance abuse, and suicide with age. Multiple animal models exposed to prenatal testosterone mirror the adverse outcomes seen in CAH. These unresolved clinical issues in CAH can lead to morbidity and cost to the U.S. healthcare system, with no effective prevention or treatment strategies.

Although CAH has been studied as a natural human model of prenatal androgen exposure to understand sexual differentiation and gendered behavior better, the prenatal phenotype remains challenging to characterize with clinical biomarkers. This situation leaves a gap in understanding the critical link between prenatal exposures and lifespan outcomes in CAH. An accurate, non-invasive method to detect and analyze the effects of hormone exposure on fetal programming would allow clinicians to characterize the severity of exposure and tailor treatment from early life.

Accordingly, there is a need for systems for collecting and analyzing images collected from a subject in a clinical setting.

### SUMMARY

In at least one aspect, a mobile, low-cost camera system for capturing face data (color, near-infrared, and depth) suitable for medical applications is provided. The proposed system is similar to the system used in the Biometric Authentication with a Timeless Learner (BATL) project, as presented in the shared paper.

In another aspect, the camera system allows for the rapid detection of facial features and data-driven analysis of the effect of early hormone exposure on facial morphology in CAH. In this regard, deep neural learning can uncover dysmorphology in patients with more subtle facial features, which can be studied longitudinally as a phenotypic biomarker and expand our understanding of lifespan outcomes in CAH.

In another aspect, artificial intelligence (AI)-based face-processing technology is applied to identify facial anomalies in subjects, and in particular, youths with CAH. Existing 3D facial capture and modeling technology is expensive and inaccessible to most clinics. This platform can efficiently capture 3D images and assess facial morphology in CAH. It is hypothesized that youth with CAH have distinctly different facial features than controls, leading to a higher facial dysmorphism score (FDS) that correlates with markers of clinical severity and androgen exposure.

In yet another aspect, a camera system for collecting image data in a clinical environment includes a camera configured to acquire color (RGB), near-infrared, and depth image data and a plurality of LED modules held proximate to the camera such that the LED modules are configured to illuminate a subject while image data is collected by the camera. The camera system also includes a microcontroller in electrical communication with the camera and the plurality of LED modules. The microcontroller is configured to actuate the plurality of LED modules and the camera such that the camera acquires the image data. A computing device is in electrical communication with the camera and the microcontroller.

In still another aspect, a camera system for collecting image data in a clinical environment includes a camera configured to acquire color (RGB), near-infrared, and depth image data and a plurality of LED modules held proximate to the camera such that the LED modules are configured to illuminate a subject while image data is collected by the camera. The camera system also includes a microcontroller in electrical communication with the camera and the plurality of LED modules. The microcontroller is configured to actuate the plurality of LED modules and the camera such that the camera acquires the image data. A computing device is in electrical communication with the camera and the microcontroller. Advantageously, the computing device is configured to execute a neural network applied to images collected by the camera system to determine a subjects' disease state.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Schematic of a camera system for collecting image data in a clinical environment with the housing removed.

FIG. 1B. Schematic of a camera system for collecting image data in a clinical environment with the housing included.

FIG. 2. Schematic of a convolutional neural network.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4. . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

For any device described herein, linear dimensions and angles can be constructed with plus or minus 50 percent of the values indicated rounded to or truncated to two significant figures of the value provided in the examples. In a refinement, linear dimensions and angles can be constructed with plus or minus 30 percent of the values indicated rounded to or truncated to two significant figures of the value provided in the examples. In another refinement, linear dimensions and angles can be constructed with plus or minus 10 percent of the values indicated rounded to or truncated to two significant figures of the value provided in the examples.

The term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “one or more” means “at least one,” and the term “at least one” means “one or more.” The terms “one or more” and “at least one” include “plurality” as a subset.

When a computing device is described as performing an action or method step, it is understood that the computing devices are operable to perform the action or method step typically by executing one or more lines of source code. The actions or method steps can be encoded onto non-transitory memory (e.g., hard drives, optical drive, flash drives, and the like).

The term “computing device” refers generally to any device that can perform at least one function, including communicating with another computing device. In a refinement, a computing device includes a central processing unit that can execute program steps and memory for storing data and a program code. .

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media (e.g., non-transitory media) such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

It should be appreciated that in any figures for electronic devices, a series of electronic components connected by lines (e.g., wires) indicates that such electronic components are in electrical communication with each other. Moreover, when lines directed connect one electronic component to another, these electronic components can be connected to each other as defined above.

Throughout this application, where publications are referenced, the disclosures of these publications in their entireties are hereby incorporated by reference into this application to more fully describe the state of the art to which this invention pertains.

The term “neural network” refers to a machine learning model that can be trained with training input to approximate unknown functions. In a refinement, neural networks include a model of interconnected digital neurons that communicate and learn to approximate complex functions and generate outputs based on a plurality of inputs provided to the model.

Abbreviations:

“AI” means artificial intelligence.

“CAH” means congenital adrenal hyperplasia.

“FSD” means facial dysmorphism score.

“LED” means light emitting diode.

“PCB” means printed circuit board.

“RGB” means red, green, blue.

The present invention provides a camera system that can be used in a clinical environment to analyze the human face. In this context, “clinical environment” means a medical setting or any situation where subjects are evaluated. The human face contains a wealth of information, including health status and clear sex differences in facial features. Brain and face morphology have been linked to conditions involving early fetal programming, such as fetal alcohol syndrome, although there is little known about the facial phenotype in patients with CAH. Recent advances in machine learning and artificial neural networks have shown great promise in analyzing and modeling human faces, revolutionizing problems related to facial analysis such as age and sex classification, emotion recognition, and person verification. In particular, the camera system set forth herein can apply deep neural networks to detect the effects of hormone abnormalities on the face in CAH.

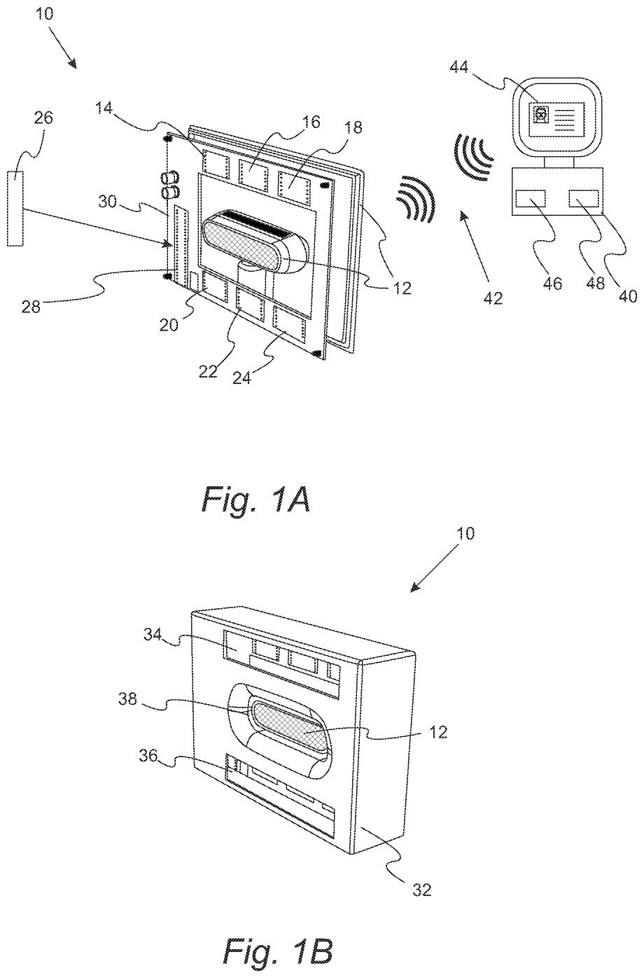

Referring to FIGS. 1A and 1B, schematics of a camera system for collecting image data in a clinical environment are provided. Camera system 10 includes a camera 12 configured to acquire color (RGB), near-infrared, and depth image data. An example of a useful camera is the Intel RealSense D435 camera [3]. The camera system 10 includes a plurality of custom-made LED modules 14-24 held proximate to the camera such that the LED modules are configured to illuminate a subject while image data is collected by the camera 10. In a refinement, each of LED modules 14-24 includes a plurality of white light LEDs, the intensity of which can be controlled by the user. A microcontroller 26 (e.g., Arduino-based microcontroller (Teensy 3:6 [1]) is in electrical communication with the camera and the plurality of LED modules. Microcontroller 26 is configured to actuate the plurality of LED modules 14-24 and the camera 12 such that the camera acquires the image data. In a refinement, camera system 10 also includes a custom-made PCB board 30 for mounting the microcontroller 26 and LED modules 14-24. In a further refinement, microcontroller 26 is positionable in microcontroller slot 28 which is mounted on PCB board. In a variation, camera system 10 also includes a housing 32 for holding the camera, the plurality of LED modules, and the microcontroller. Housing 32 defines slots 34 and 36 that allow light to pass therethrough and slot 38 that allows the camera to collect image data. In a refinement, housing 32 is custom-made by 3D printing. Advantageously, artificial intelligence techniques can be applied to images collected by camera system 10 to evaluate subj ects having or suspected of having congenital adrenal hyperplasia.

In a variation, camera system 10 further includes computing device 40, which is in electrical communication with the camera 12 and the microcontroller 26 via wireless or wired connection 42. Typically, computing device 40 is configured to execute a control program for the camera system. In a refinement, computing device 40 is configured to interact with a user through a graphical user interface 44 implemented by graphical user interface software (e.g., Custom-made Python graphical User Interface (GUI) software) executing on computing device 40. The graphical user interface software can be configured for capturing and storing data. Advantageously, the computing device 40 is configured to send a capture request to the camera and the microcontroller such that all LEDs flash at a predetermined brightness. The camera captures data from a subject sitting in front of the system. In a refinement, computing device 40 includes a data storage component 46 for storing the image data in electrical communication with a computer processor 48 that can be configured to implement the method described herein.

Advantageously, the camera system 10 is configured for capturing face data (color, near-infrared, and depth) suitable for medical applications. In a refinement, the camera system is configured for the rapid detection of facial features and data-driven analysis of the effect of early hormone exposure on facial morphology in CAH. In a further refinement, the camera system is configured to allow the determination of the dysmorphism score (FDS) that correlates with markers of clinical severity and androgen exposure, particularly in subjects suspected of having CAH.

In another variation, deep neural learning is applied to the images collected by camera system 10 to uncover dysmorphology in patients with more subtle facial features. In this regard, images constructed from the image data collected by the camera system are classified by a machine learning algorithm executing on the computing device 10 into a plurality of predetermined classifications. In a refinement, images constructed from the image data collected by the camera system are classified by a trained neural network. Typically, the trained neural network is trained with characterized images collected by the camera system.

In a refinement, computing device 40 applies a convolutional neural network to learn a complex mapping function to classify images generated by the camera system. The convolutional neural network is trained with a test set derived from characterized images. A subject's imaging data or test image data is provided as input to the convolutional neural network. The trained convolutional neural network is applied to a subject's imaging data and then classified. It should be appreciated that the convolutional network can include convolutional layers, pooling layers, fully connected layers, normalization layers, a global mean layer, a batch-normalization layer, and the like.

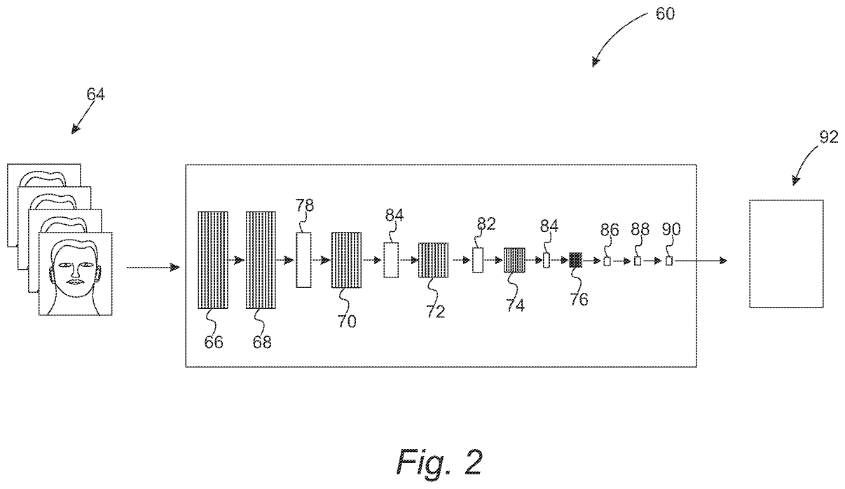

With reference to FIG. 2, an idealized schematic illustration of a convolutional neural network executed by computing device 40 or another computing device is provided. It should be appreciated that any deep convolutional neural network that operates on digitized image data and/or pre-processed input image data can be utilized. The convolutional network 60 can include convolutional layers, pooling layers, fully connected layers, normalization layers, a global mean layer, and a batch-normalization layer. Batch normalization is a regularization technique, which may also lead to faster learning. Convolutional neural network layers can be characterized by sparse connectivity where each node in a convolutional layer receives input from only a subset of the nodes in the next lowest neural network layer. The convolutional neural network layers can have nodes that may or may not share weights with other nodes. In contrast, nodes in fully connected layers receive input from each node in the next lowest neural network layer. For both convolutional layers and fully connected layers, each node calculated its output activation from its inputs, weight, and an optional bias.

An untrained neural network (e.g., an untrained convolutional neural network) is trained with a training set to form a trained neural network (e.g., a trained convolutional neural network). During training, optimal values for the weight and bias are determined. For example, convolutional neural network 60 can be trained with a training set of data 64 that includes a plurality of images from a subject that has been annotated and/or classified (e.g. by hand) by an expert. Typically, the images are digitized prior to being input to the neural network. in a refinement, the digital images in the training set can be classified with respect to a disease state (e.g., CAH), age, sex, emotional status (e.g., emotion recognition), and personal identity (e.g., person verification). The classification with respect to a disease state can be whether or not the subject has a predetermined disease state. In some variations, convolutional neural network 60 is trained with a training set that includes images of subjects known to have dysmorphology (e.g., subjects with CAH) and images of subjects without such dysmorphology. Such images are identified by an expert (e.g., a medical doctor).

After training, the trained neural network can be used to identify and/or classify images from a subject of unknown status to classify the subjects of unknown status with respect to disease state, disease state (e.g., CAH), age, sex, emotional status (e.g., emotion recognition), personal identity (e.g., person verification) and/or any other predetermined classification.

Referring to FIG. 2, convolutional neural network 60 includes convolution layers 66, 68, 70, 72, 74, and 76 as well as pooling layers 78, 80, 82, 84, and 86. The pooling layers can be a max pooling layer or a mean pooling layer. Another option is to use convolutional layers with a stride size greater than 1. FIG. 2 also depicts a network with global mean layer 88 and batch normalization layer 90. The present embodiment is not limited by the number of convolutional layers, pooling layers, fully connected layers, normalization layers, and sublayers therein. The output (e.g., classifications) of the neural network is provided as output 92.

Additional details of the camera system for collecting image data in a clinical environment and components included therein is found in L. Spinoulas et al., “Multispectral Biometrics System Framework: Application to Presentation Attack Detection,” in IEEE Sensors Journal, vol. 21, no. 13, pp. 15022-15041, 1 Jul. 1, 2021, doi: 10.1109/JSEN.2021.3074406 and in International Patent Application PCT/US22/33191; the entire disclosures of which are hereby incorporated by reference in their entirety.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

### REFERENCES

[1] “Teensy 3.6,” www.pjrc.com/store/teensy36.htm1.

[2] “OSRAM, White LED Indication-Discrete 3.2V 2-PLCC,” www. digikey.com/product-detail/en/osram-opto-semiconductors-inc/LW-T67C-S2V1-5K8L-0-20-R18-Z/475-2544-1-ND/1802671.

[3] “Intel RealSense™ Depth Camera D435,” https://www.intelrealsense.com/depth-camera-d435/.

What is claimed is:

1. A camera system for collecting image data in a clinical environment, the camera system comprising:
a camera configured to acquire color (RGB), near-infrared, and/or depth image data;
a plurality of LED modules positioned proximate to the camera such that the plurality of LED modules is configured to illuminate a subject while image data is collected by the camera;
a microcontroller in electrical communication with the camera and the plurality of LED modules, the microcontroller configured to actuate the plurality of LED modules and the camera such that the camera acquires the image data; and
a computing device in electrical communication with the camera and the microcontroller.

2. The camera system of claim 1, wherein the computing device is configured to execute a control program for the camera system.

3. The camera system of claim 1, wherein the computing device is configured to interact with a user through a graphical user interface implemented by graphical user interface software.

4. The camera system of claim 3, wherein graphical user interface software is configured for capturing and storing data.

5. The camera system of claim 3 wherein the computing device is configured to sends a capture request to the camera and the microcontroller such that all LEDs flash at a predetermined brightness.

6. The camera system of claim 5, wherein the computing device includes a data storage component for storing the image data.

7. The camera system of claim 5, wherein images constructed from the image data collected by the camera system are classified by a machine learning algorithm executing on the computing device.

8. The camera system of claim 5, wherein images constructed from the image data collected by the camera system are classified by a trained neural network.

9. The camera system of claim 8, wherein the trained neural network is trained with characterized image collected by the camera system.

10. The camera system of claim 1, wherein the plurality of LED modules and the microcontroller are mounted on a PCB board.

11. The camera system of claim 1, wherein the plurality of LED modules includes white light LEDs, intensity of which are user-controllable.

12. The camera system of claim 1 further comprising a housing for holding the camera, the plurality of LED modules, and the microcontroller.

13. The camera system of claim 1, wherein artificial intelligence techniques are applied to images collected by the camera system to evaluate a subject having or suspected of having congenital adrenal hyperplasia.

14. The camera system of claim 1, wherein a trained neural network is applied to images collected by the camera system to evaluate subjects having or suspected of having congenital adrenal hyperplasia.

15. The camera system of claim 14, wherein the trained neural network is a trained convolutional neural network.

16. The camera system of claim 1, wherein the computing device is configured to execute a trained neural network to determine a subject's disease state.

17. The camera system of claim 16, wherein the computing device is configured to execute the trained neural network to determine if a subject has congenital adrenal hyperplasia.

18. A camera system for collecting image data in a clinical environment, the camera system comprising:
a camera configured to acquire color (RGB), near-infrared, and/or depth image data;
a plurality of LED modules positioned proximate to the camera such that the plurality of LED modules is configured to illuminate a subject while image data is collected by the camera;
a microcontroller in electrical communication with the camera and the plurality of LED modules, the microcontroller configured to actuate the plurality of LED modules and the camera such that the camera acquires the image data; and
a computing device in electrical communication with the camera and the microcontroller, the computing device configured to execute a trained neural network applied to images collected by the camera system to determine a subject's disease state.

19. The camera system of claim 18, wherein the trained neural network is a trained convolutional neural network.

20. The camera system of claim 17, wherein the computing device is configured to sends a capture request to the camera and the microcontroller such that all LEDs flash at a predetermined brightness.
